(19) United States
(12) Patent Application Publication
(10) Pub. No.: US 2022/0001053 A1
(43) Pub. Date: Jan. 6, 2022

(54) DRAWER STERILIZATION SYSTEM

(71) Applicant: Gael Lambert, Princeton, NJ (US)

(72) Inventor: Gael Lambert, Princeton, NJ (US)

(21) Appl. No.: 17/366,294

(22) Filed: Jul. 2, 2021

Related U.S. Application Data
(60) Provisional application No. 63/047,729, filed on Jul. 2, 2020.

(51) Int. Cl. A61L 2/10; A47B 88/919

(52) U.S. Cl. CPC A61L 2/10; A47B 88/919; A61L 2202/14; A61L 2202/122; A61L 2202/11

(57) ABSTRACT

A kit for retrofitting an existing drawer with a sterilization system, that utilizes one or more light sources configured to emit UV-C light positioned internally within a drawer casing, the light sources being connected through a switch to a power supply. The kit contains components that can be configured such that when the drawer is closed, the switch closes, causing the light source(s) to turn on and sterilize the contents of the drawer.

### CROSS-REFERENCE TO RELATED APPLICATIONS

This application claims priority to U.S. Provisional Pat. App. No. 63/047,729, filed Jul. 2, 2020, which is herein incorporated by reference in its entirety.

### BACKGROUND

Modern civilization relies on drawers to store many items, including clothing, food, and physical currencies (including coins and paper bills). It is known that viruses, fungi, or bacteria can survive, e.g., on the items stored in drawers (such as currency) for a sufficient length of time to allow the virus, fungi, or bacteria to transfer to another person.

Many sterilization methods require a person to take additional steps beyond their normal patterns of behavior, and such steps can sometimes be forgotten. Still other sterilization methods require special configurations, such as specific chemicals or a high degree of ventilation.

Therefore, what is needed is a way to retrofit an existing drawer, that results in a drawer does not require a person to take additional steps in order to sterilize any items placed in the drawer.

### BRIEF SUMMARY

The present disclosure is a kit for retrofitting an existing drawer with a sterilization system. The kit generally comprises at least four components: (i) at least one light source, (ii) a first switch, (iii) a timer module, and (iv) wiring.

The light source(s) are configured to be attached to the top internal surface of a drawer casing, within an internal cavity that is defined by the internal surfaces of the drawer casing (e.g., cavity that the drawer enters into is generally defined by the top, back, sides, and bottom of the drawer casing). The light source(s) are configured to emit at least one ultraviolet (UV) wavelength, and specifically UV-C wavelengths, towards a drawer when the drawer is inserted into the internal cavity of the drawer casing.

The first switch is also configured to be attached to one of the internal surface of the drawer casing, and is configured to turn the light source(s) on when the drawer is closed and turn the light source(s) off when the drawer opens.

The timer module is configured to control how long the light source(s) emit the at least one UV wavelength after it is turned on. That is, once the light source(s) turn on, the timer module is configured to wait for a certain period of time, and then turn off light source(s) or at least stop emitting the UV wavelength(s).

The wiring is adapted to connect a power supply to the first switch, the timer module, and the at least one light source.

Preferably, neither the drawer nor the drawer casing comprises a ventilation aperture.

Preferably, the kit also includes a magnet that is configured to be placed on an internal surface of the drawer such that the magnet will close the first switch when the drawer is closed.

Optionally, the kit also includes at least one external visual indicator in electrical communication with the first switch, the visual indicator configured to provide a visual indication of when the light source(s) are emitting UV wavelength(s).

Optionally, the kit also includes at least one additional manual switch configured to prevent the light source(s) from emitting the UV wavelength(s) when the switch is open.

Optionally, the wiring is configured with one or more connectors to allow various components to be removably attached to the wiring. For example, in some embodiments, the wiring is configured such that that the light source(s) can be removably connected to the wiring, and/or the wiring is configured with one or more connectors such that an external power source (e.g., a cord connected to an AC outlet in a home) or internal power source (e.g., a battery or supercapacitor) can be removable connected to the wiring.

Optionally, the timer module may comprise a processor, and/or a display. Optionally, the processor is configured with instructions that, when executed, allow a user to adjust how long the light source(s) emit the UV wavelength(s) after it is turned on.

In one embodiment, the kit consists of (i) the light source(s); (ii) the first switch; (iii) the timer module; (iv) an electrical connector to an external power source (such as a cord with a plug for inserting into a standard AC outlet), (v) the wiring, where the first switch and the timer module are directly attached to the wiring, and the wiring optionally directly attaches to one or more connectors which are configured to allow the at least one light source, the electrical connector, or both to be removably attached to the wiring; (vi) a magnet configured to be placed on an internal surface of the drawer such that the magnet will close the first switch when the drawer is closed; (vii) optionally at least one external visual indicator in electrical communication with the first switch, the visual indicator configured to provide a visual indication of when the at least one light source is emitting the at least one UV wavelength; and (viii) optionally at least one additional manual switch configured to prevent the at least one light source from emitting the at least one UV wavelength when the switch is open.

### BRIEF DESCRIPTION OF THE DRAWINGS

FIG. 1 is a depiction of an embodiment of a disclosed retrofitting kit in place within a drawer casing/drawer unit.

FIG. 2 is a depiction of an alternate embodiment of a disclosed kit.

### DETAILED DESCRIPTION

Disclosed is a retrofitting kit for retrofitting an existing drawer (such as a cash drawer, a clothes drawer, etc.) with a sterilization system, and a UV-C sterilization system in particular, in order to effectively eliminate virus, fungi, and/or bacteria from items (such as currency, clothes, food, etc.) placed into the drawer. The disclosed easy-to-install kits provide simple (only provides for powering and controlling UV light sources, with optional visual indicators) sterilization that is safe and effective.

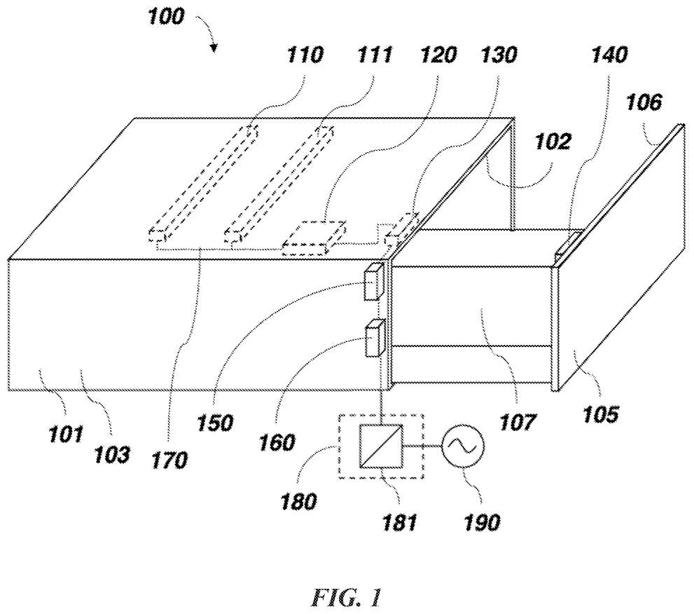

Referring to FIG. 1, one embodiment of an installed retrofitting kit (100) can be seen. The retrofitting kit comprises, consists essentially of, or consists of, one or more light sources (110, 111) positioned internally within a drawer casing (101), where the light source(s) are electrically connected via wiring (170) through a switch (130) to a power supply (180), such that when the drawer (105) is closed, the switch (130) is closed, causing the light source(s) (110,111) to turn on for a period of time controlled by the timer module (120). Other components may be included, but only if the additional components do not materially alter the basic and novel characteristics the claimed retrofitting kit.

The one or more light sources (110, 111) may be any UV light source, and is preferably a UV-C light source, and more preferably configured to emit at a peak wavelength between about 200 nm and about 300 nm, and more preferably in the 207 nm to 222 nm range and/or in the 250 nm to 280 nm range. In some embodiments, where two or more light sources (110, 111) are provided, each light source emits the same range of wavelengths. In some embodiments, two or more light sources (110, 111) are provided, at least one light source (110) emits a different range of wavelengths than at least one other light source (111). The light source(s) may be a single emitter, multiple emitters, or an array of emitters. In embodiments utilizing more than one emitter, the emitters may be in series or in parallel.

The one or more light sources (110, 111) are configured to be positioned within the internal cavity of the drawer casing defined by the various internal surfaces of the drawer casing (e.g., typically, the top, bottom, back, left side, and right sides). The one or more light sources (110, 111) are configured to preferably be attached to a top internal surface (102) of the drawer casing (101) and are configured to emit UV light towards a drawer (105) that slides or is otherwise capable of being inserted into the internal cavity of the drawer casing. Preferably, the light source(s) are configured to emit UV light downward towards a bottom portion (107) of the drawer (105).

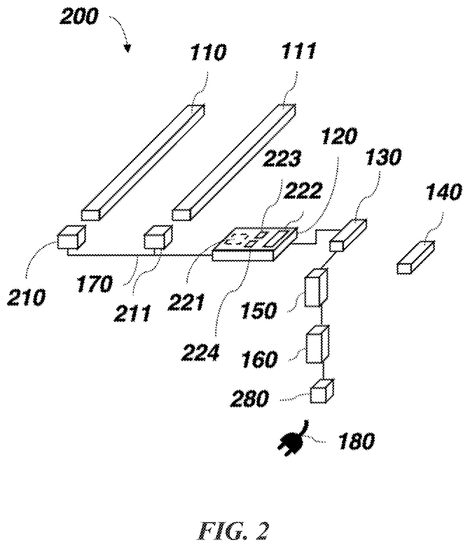

In FIG. 1, the light sources (110, 111) are directly attached to the wiring (170). Referring briefly to FIG. 2, in some embodiments, the wiring (170) is attached to one or more light source connectors (210, 211), and that the light sources (110, 111) are removably attached to the wiring (170) through the light source connectors (210, 211).

The switch (130) may be any appropriate switch for a closing drawer, and may require an additional component (for example, magnet (140)) to function. In FIG. 1, the switch (130) is a normally-open reed switch, and when the drawer is shut, the magnet (140) causes the connection to close, and the light source to activate. The function of the switch is to turn the light source(s) on when the drawer closes and turn the light source(s) off when the drawer opens. The switch will preferably be positioned near the front opening defined by the casing (101), the opening through which the drawer (105) will be inserted. The switch will generally be within the internal cavity of the drawer casing, attached to the top, either side, or bottom, near the opening, although preferably it is position on the top. In some embodiments, it is positioned on the top inner surface (102), no more than a third of the width of the drawer away from the left or right internal surface of the drawer casing.

The wiring (170) is generally comprised of any insulated conductive material known to those of skill in the art. The wiring is adapted to connect a power supply (180) located external to the drawer casing and/or within the internal cavity, to the first switch, the timer module, and the at least one light source.

The power supply (180) can be any appropriate power supply. It may, for example, simply be a cord that is directly connected to a power source (190), such as batteries, solar cells, or a building's electrical outlets. Referring briefly to FIG. 2, in some embodiments, the wiring (170) may contain a power supply connector (280) that allows the power supply (180) to be removably connected to the wiring (170).

Referring back to FIG. 1, in some embodiments, the power supply (180) may optionally be removable from both the wiring (170) and power source (190), and may contain, e.g., a power converter (181) (such as an AC to DC power converter), and/or other necessary components as understood by those of skill in the art for ensuring that the power being provided by the power source (190) is adjusted to the appropriate current, voltage, etc., as required by the other components in the kit (such as the light source(s) (110, 111), the timing module (120), etc.).

The timing module (120) controls how long each light source (110, 111) emits UV light after the drawer is closed. The timer module (120) may be configured to provide a programmable or non-programmable timer function. Referring briefly to FIG. 2, the timing module (120) may comprise, consist essentially of, or consist of a processor (221), electric circuitry (not shown), optionally a display (222), and optionally one or more buttons (223,224).

In some embodiments, the one or more light sources (110, 111) emit continuously for a predetermined period of time. For surface disinfection, the dosage provided is equal to the irradiance multiplied by time. The dose-response curves for various microorganisms are well-known, or easily determined. For example, it is known that a UV-C dose of just 3.7 mJ/cm² is sufficient to achieve a more than 3-log inactivation of the SARS-CoV-2 virus. Thus, the predetermined period of time can be determined by dividing the required dosage for a target log inactivation of a microorganism by an estimate of the effective irradiance for the specific light source. For example, if an LED operated at 350 mA, at a distance of about 6 cm from the surface to be sterilized has an effective irradiance of about 0.075 mW/cm², the required time to provide a 3-log inactivation of SARS-CoV-2 virus is just over 49 seconds (3.7 mJ/cm²/0.075 mW/cm²=49.33 seconds).

In some embodiments, the light sources are configured to provide sufficient irradiance that the predetermined period of time the lights remain active is, e.g., ≤20 minutes, ≤15 minutes, ≤10 minutes, ≤5 minutes, ≤2 minutes, ≤1 minute, ≤50 seconds, or ≤30 seconds.

In some embodiments, the amount of time is not predetermined based on a target degree of inactivation of a microorganism. The light sources simply begin irradiating when the drawer is shut, and continue to attempt to inactivate microorganisms until the light stops, regardless of how many microorganisms are inactivated. In such embodiments, the timer module allows for an adjustable or non-adjustable predetermined maximum period of time that the light will remain active if the drawer remains shut. In some embodiments, the predetermined maximum period of time the lights remain active is, e.g., ≤30 minutes, ≤25 minutes, ≤20 minutes, ≤15 minutes, ≤10 minutes, or ≤5 minutes.

In some embodiments, where the drawers are expected to remain closed for longer periods of time, it may be advantageous to reduce the power requirements of the light sources, such that the predetermined period of time may be extended, such up to 240, 120, or 60 minutes.

In preferred embodiments, the at least one processor is configured with instructions that, when executed, allow a user to adjust how long the at least one light source emits the at least one UV wavelength after it is turned on.

Referring briefly to FIG. 2, in some embodiments, the timing module (120) includes a processor configured with instructions that, when executed, allow it to receive input from one or more buttons (223, 224), and based on the received input, adjust the delay after the drawer is closing before the light source(s) activate (in some embodiment, this defaults to zero, i.e., no delay), adjust the time the light source(s) emits UV light, adjust the power provided to the light source(s), and/or adjust the dosage to be provided. In some cases, such variables are all independently controllable. In other embodiments, at least one variable is fixed, and other variables are automatically adjusted as necessary. For example, in some embodiments, the dosage is fixed, such that if the user adjusts the irradiation time, the processor contains instructions that, when executed, causes the power being sent to the light sources to be adjusted, to ensure the dosage remains constant. In other embodiments, the irradiance of the light sources is fixed, such that if the user adjusts a dosage, the amount of time is automatically adjusted.

Referring back to FIG. 1, the drawer (105) can be any appropriate drawer. In some embodiments, the drawer is configured to be removed from the casing (101). In some embodiments, if a magnet (140) is used to, the magnet is attached to a front portion of the drawer, such as an inner surface of a front portion (106) of the drawer. In some embodiments, the drawer is configured to slide into and out of the casing on rollers or rails (not shown). In some embodiments, the drawer comprises only a bottom portion (107), but no front portion (106). For example, in some embodiments, the casing (101) has a front door (not shown) that is attached via a hinge to the rest of the casing, the magnet (140) may be attached to the front door, and the drawer (105) that only has a bottom portion (107) can be slid out of the casing (101) when the front door is open.

In some embodiments, the drawer contains no internal partitioning or other configurations.

The casing (101) can be comprised of any appropriate material. Preferably, the casing (101) is comprised of a UV-opaque material (i.e., there is substantially zero transmission of UV light through the casing).

In preferred embodiments, neither the drawer casing (101) nor the drawer (105) has apertures for ventilation.

Optionally, the kit may include a magnet (140) configured to be placed on an internal surface (106) of the drawer (105) such that the magnet will close the first switch (130) when the drawer is closed.

Optionally, the kit may include one or more manual switches (160) on an external surface (103) of the drawer casing (101). The manual switches are in electrical communication with the power supply and the one or more light sources, such that the manual switches can prevent the light source from activating if the manual switches are not closed.

Optionally, the kit may also include one or more visual indicators (150) on an external surface (103) of the drawer casing. In some embodiments, the visual indicators may indicate when the light source is active, when the kit is connected to a power supply, and/or when a manual switch (160) is turned on. In some embodiments, the lights are positioned on the left or right side of the casing, or on exterior of a front door or front portion of the drawer.

Referring to FIG. 2, it is seen that, in some embodiments, a kit (200) comprises, consists essentially of, or consists of: (i) the at least one light source (110, 111); (ii) the first switch (130); (iii) the timer module (120); (iv) the power supply (180); (vi) the wiring (170), wherein the first switch (130) and the timer module (120) are directly attached to the wiring, the wiring optionally directly attached to one or more connectors (210, 211, 280) configured to allow the at least one light source, the power supply, or both to be removably attached to the wiring; (vii) a magnet (140) configured to be placed on an internal surface of the drawer such that the magnet will close the first switch (130) when the drawer is closed; (viii) optionally at least one external visual indicator (150) in electrical communication with the first switch, the visual indicator configured to provide a visual indication of when the at least one light source is emitting the at least one UV wavelength; and (ix) optionally at least one additional manual switch (160) configured to prevent the at least one light source from emitting the at least one UV wavelength when the switch is open.

What is claimed is:

1. A kit for retrofitting an existing drawer with a sterilization system, comprising:
at least one light source configured to be attached to a top internal surface of a drawer casing in an internal cavity defined by a plurality of internal surfaces of the drawer casing, the top internal surface being one of the plurality of internal surfaces, the at least one light source configured to emit at least one ultraviolet (UV) wavelength towards a drawer capable of being inserted into the internal cavity of the drawer casing;
a first switch configured to be attached to one of the plurality of internal surface of the drawer casing to turn the at least one light source on when the drawer is closed and turn off when the drawer opens;
a timer module configured to control how long the at least one light source emits the at least one UV wavelength after it is turned on;
a power supply; and
wiring adapted to connect the power supply to the first switch, the timer module, and the at least one light source.

2. The kit according to claim 1, wherein neither the drawer nor the drawer casing comprises a ventilation aperture.

3. The kit according to claim 1, further comprising a magnet configured to be placed on an internal surface of the drawer such that the magnet will close the first switch when the drawer is closed.

4. The kit according to claim 1, further comprising at least one external visual indicator in electrical communication with the first switch, the visual indicator configured to provide a visual indication of when the at least one light source is emitting the at least one UV wavelength.

5. The kit according to claim 1, further comprising at least one additional manual switch configured to prevent the at least one light source from emitting the at least one UV wavelength when the switch is open.

6. The kit according to claim 1, wherein the wiring is configured with one or more light source connectors such that the one or more light sources can be removably connected to the wiring.

7. The kit according to claim 6, wherein the wiring is configured with one or more power supply connectors such that the power supply can be removable connected to the wiring.

8. The kit according to claim 1, wherein the timer module comprises at least one processor.

9. The kit according to claim 8, wherein the timer module further comprises a display.

10. The kit according to claim 9, wherein the at least one processor is configured with instructions that, when executed, allow a user to adjust how long the at least one light source emits the at least one UV wavelength after it is turned on.

11. The kit according to claim 1, wherein the kit consists of:
the at least one light source;
the first switch;
the timer module;
the power supply;
the wiring, wherein the first switch and the timer module are directly attached to the wiring, the wiring optionally directly attached to one or more connectors configured to allow the at least one light source, the power supply, or both to be removably attached to the wiring;
a magnet configured to be placed on an internal surface of the drawer such that the magnet will close the first switch when the drawer is closed;
optionally at least one external visual indicator in electrical communication with the first switch, the visual indicator configured to provide a visual indication of when the at least one light source is emitting the at least one UV wavelength; and
optionally at least one additional manual switch configured to prevent the at least one light source from emitting the at least one UV wavelength when the switch is open.
